(19) United States
(12) Patent Application Publication
(10) Pub. No.: US 2021/0241120 A1
(43) Pub. Date: Aug. 5, 2021

(54) SYSTEMS AND METHODS FOR IDENTIFYING SYNTHETIC IDENTITIES

(71) Applicant: Experian Information Solutions, Inc., Costa Mesa, CA (US)

(72) Inventors: Kevin Chen, San Diego, CA (US); Mason L. Carpenter, Richmond, VA (US); Yi He, San Diego, CA (US); Hua Li, San Diego, CA (US); Zhixuan Wang, San Diego, CA (US); Christer DiChiara, Carlsbad, CA (US); Sophie Liu, San Diego, CA (US); Eric Haller, Encinitas, CA (US); Shanji Xiong, San Diego, CA (US); Honghao Shan, San Diego, CA (US); Liang Lin, San Diego, CA (US); Brian Duke, Poway, CA (US); Chi Zhang, San Diego, CA (US); Doris Wang, San Diego, CA (US); Seth Kressin, Carlsbad, CA (US)

(21) Appl. No.: 17/161,525

(22) Filed: Jan. 28, 2021

Related U.S. Application Data
(60) Provisional application No. 62/968,098, filed on Jan. 30, 2020.

(51) Int. Cl. G06N 3/08; G06N 3/04

(52) U.S. Cl. CPC G06N 3/088; G06N 3/0454

(57) ABSTRACT

Systems and methods are provided for implementing machine learning techniques to distinguish a real identity, such as a set of identity information representing a real person, from a synthetic identity that may include portions of real identity information. Attributes regarding a target identity derived from a variety of retrieved data records may be provided as input to multiple machine learning models that generate scores associated with the potential of the target identity being synthetic. The scores may be combined and compared to a threshold to generate a determination of whether the target identity is a synthetic identity.

### PRIORITY AND INCORPORATION BY REFERENCE

This application claims benefit of U.S. Provisional Patent Application No. 62/968,098, entitled “SYSTEMS AND METHODS FOR IDENTIFYING SYNTHETIC IDENTITIES,” filed Jan. 30, 2020, which is hereby incorporated by reference in its entirety.

### BACKGROUND

### Field of the Invention

The present disclosure relates to generating and/or implementing database systems that enable a determination of whether a particular entity is a legitimate entity or a synthetic entity based on an analysis of records from a plurality of selectable sources.

### Description of the Related Art

Current database systems may not be able to easily identify a synthetic entity from a legitimate entity in a majority of instances. For example, such current database systems may be unable to accurately and consistently identify synthetic entities when the synthetic entities are associated with records using information from, associated with, or appearing to be associated with legitimate entities (for example, a legitimate entity's name, social security number, and so forth, combined with false information that may collectively appear to be a real identity record in a database). However, knowledge of a synthetic entity may be beneficial to limiting liability during or after transactions. For example, if a current system receives a request from the particular entity to open a credit card, the system is unaware whether the particular entity is legitimate or synthetic. Thus, the system is unable to generate an output (for example, a flag, a report, an alert, and so forth) indicating that that the particular entity is a synthetic entity. Thus, the system may be unable to help reduce risk and/or liability associated with particular entity. Improved systems, devices, and methods for efficiently and effectively identifying synthetic entities based on databases with billions of records including varying information between different records and where various rules and/or restrictions regarding identifying synthetic entities are desired.

### BRIEF DESCRIPTION OF THE DRAWINGS

FIG. 1 shows a network diagram of various components that communicate via a communication network and form an identity scoring platform that provides scores for entities having records processed by the identity scoring platform.

FIG. 2A shows a block diagram indicating components within the identity scoring platform of FIG. 1 that process data and apply machine learning models based on received requests.

FIG. 2B shows a flow diagram indicating data flow within the identity scoring platform of FIG. 1 based on a request to identify an identity score for a particular entity using a plurality of machine learning models.

FIG. 3 shows graphs representing how a sample of attributes described herein correlate to a likelihood of being a synthetic identity or entity.

FIG. 4 shows an example of machine learning models used by the platform of FIG. 1.

FIG. 5A is a block diagram of a first example machine learning model applied to the records to identity a first identity score.

FIG. 5B is a block diagram of a second example machine learning model applied to the records to identity a second identity score.

FIG. 6 is a block diagram showing example components of the identity scoring platform of FIG. 1.

### DETAILED DESCRIPTION

Although certain embodiments and examples are disclosed below, inventive subject matter extends beyond the specifically disclosed embodiments to other alternative embodiments and/or uses and to modifications and equivalents thereof. Thus, the scope of the application is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

### Overview

Financial institutions often receive requests from customers or entities to open a new financial account (for example, a bank, credit card, or loan account). However, because there is no centralized and/or reliable source for identifying when an identity is a real identity (for example, a person's own information) as opposed to a synthetic identity (for example, an identity defined by the applicant based in part on fictitious or fraudulent information submitting to open the new account), it is difficult for the financial institutions to identify the synthetic identities before the account is opened. Over time, credit bureau databases and other databases of personal records may become populated with records that appear to be about a real person, but are in fact about a synthetic identity that has been created by a fraudster through strategic account openings that may use a mix of real and fake information (e.g., name, address, social security number, phone number, etc.). For example, synthetic identities are often made to look like real people with good credit scores and histories, but are fabricated by fraudsters to perpetrate fraud. Such synthetic identities are often based on a Social Security number (SSN), or a credit privacy number (CPN). They are often made up of blended information which combines real and fake data, such as an address from one person mixed with another person's SSN or CPN. Furthermore, while the synthetic identity problem has existed for years, because there is no a true fraud victim, the financial institutions have difficulty separating synthetic identity fraud from credit losses associated with a truthfully named person. Synthetic identities present risks to the financial institutions because purchases and charges to accounts attributed to synthetic identities are often not reimbursed and/or otherwise written off as losses for the financial institutions.

The systems and methods described herein address this challenge by applying a novel two-layer analytical framework that is unprecedented at a time when financial institutions are demanding a solution to slow the rapidly growing impact of such synthetic identity risk. The first layer of this analytical framework applies logic establishing criteria to create a core definition to determine whether the identity is real or synthetic. The definition may be derived based on domain knowledge or supported with data analysis. The definition may be used as agreed upon criteria for reimbursement by the party guarantees to cover the loss due to synthetic identities. The definition may also be used as a pseudo target when developing and evaluating analytical models. The second layer applies artificial intelligence models and technologies and generates new insights into identity attributes to separate real identities from potential synthetic identities based on records accessible to the systems and methods.

The systems and methods described herein may utilize data from one or more selectable sources. In some embodiments, the data from the sources is aggregated into a single linked database. For example, the linked database brings together data assets across one or more business units and augments the aggregated data with additional digital identity data to provide a comprehensive view of entity identity and rich insights into identity behavior. The systems and methods may apply one or more behavioral attributes enabling identification and targeting of behaviors associated with synthetic identities as compared to real identities. These attributes examine many aspects of identity behavior, including breadth and history of an identity's footprint across different businesses, relationships of the identity to other identities, and stability and velocity of change of the identity over time. For example, attributes may correspond to characteristics of records that show a correlation between records in the data stores and corresponding risk of being synthetic. For example, one or more records in the data stores 104 and 108 include address information. One attribute may relate to how many records share a particular address. For example, some addresses may not be shared between records (for example, for an entity that had an address alone) while some addresses may be shared between multiple people (for example, an office with multiple employees or a residential address for a family of four people). Addresses that are not shared may have a slight risk of being synthetic, while addresses shared with between two and five people may not have a high risk, while addresses with more than ten people may have a highest risk of being synthetic. Thus, this attribute may show a relationship between number of entities sharing an address and a risk of being synthetic. Such attributes may strengthen the ability of the systems and methods to accurately and consistently identify synthetic identities. Further details regarding the systems and methods and the analytical framework applied by the systems and methods are provided below.

Synthetic identities are generally manufactured, not stolen. Because synthetic identities are generally formed from aggregate information, synthetic identities may be difficult to identify and may go unreported (for example, because there may not be a single or actual victim). The systems and methods described herein improve accuracy of detection of synthetic identities over existing systems, enabling financial and related institutions to reduce risk by identifying transactions with synthetic identities before the transaction or other action is completed.

### Terms

To facilitate an understanding of the systems and methods discussed herein, a number of terms are described below. The terms described below, as well as other terms used herein, should be construed broadly to include the described information, the ordinary and customary meaning of the terms, and/or any other implied meaning for the respective terms. Thus, the descriptions below do not limit the meaning of these terms, but only provide exemplary descriptions.

Data Store: Includes any computer readable storage medium and/or device (or collection of data storage mediums and/or devices). Examples of data stores include, but are not limited to, optical disks (for example, CD-ROM, DVD-ROM, and so forth), magnetic disks (for example, hard disks, floppy disks, and so forth), memory circuits (for example, solid state drives, random-access memory (“RAM”), and so forth), and/or the like. Another example of a data store is a hosted storage environment that includes a collection of physical data storage devices that may be remotely accessible and may be rapidly provisioned as needed (commonly referred to as “cloud” storage).

Database: Includes any data structure (and/or combinations of multiple data structures) for storing and/or organizing data, including, but not limited to, relational databases (for example, Oracle databases, MySQL databases, and so forth), non-relational databases (for example, NoSQL databases, and so forth), in-memory databases, spreadsheets, as comma separated values (“CSV”) files, eXtendible markup language (“XML”) files, TeXT (“TXT”) files, flat files, spreadsheet files, and/or any other widely used or proprietary format for data storage. Databases are typically stored in one or more data stores. Accordingly, each database referred to herein (for example, in the description herein and/or the figures of the present application) is to be understood as being stored in one or more data stores.

Database Record and/or Record: Includes one or more related data items stored in a database. The one or more related data items making up a record may be related in the database by a common key value and/or common index value, for example.

Output, Notification, and/or Alert: Includes any electronic notification sent from one computer system to one or more other computing systems. For example, a notification may indicate details or a response to a request. Notifications may include information regarding the request, the response, score information, and so forth and may indicate, for example, to a user, the result of the request. Notifications may be transmitted electronically, and may cause activation of one or more processes, as described herein.

User: depending on the context, may refer to a person, such as an individual, consumer, or customer, and/or may refer to an entity that provides input to the system and/or an entity that utilizes a device to receive the output, notification or alert (for example, a user who is interested in identifying whether an identity is synthetic or real). Thus, in the first context, the terms “user,” “individual,” “consumer,” and “customer” should be interpreted to include single persons, as well as groups of users, such as, for example, married couples or domestic partners, organizations, groups, and business entities and institutions. Additionally, the terms may be used interchangeably. In some embodiments, the terms refer to a computing device of a user rather than, or in addition to, an actual human operator of the computing device.

Requesting Entity generally refers to an entity, such as a person, business, a non-profit organization, an educational institution, a financial institution, etc., that requests information and/or services from one or more of the systems or platforms discussed herein. For example, a requesting entity may comprise a financial institution that wants to determine whether a target identity is synthetic or real before completing or authorizing a transaction or action with the target identity.

### Identity Platform

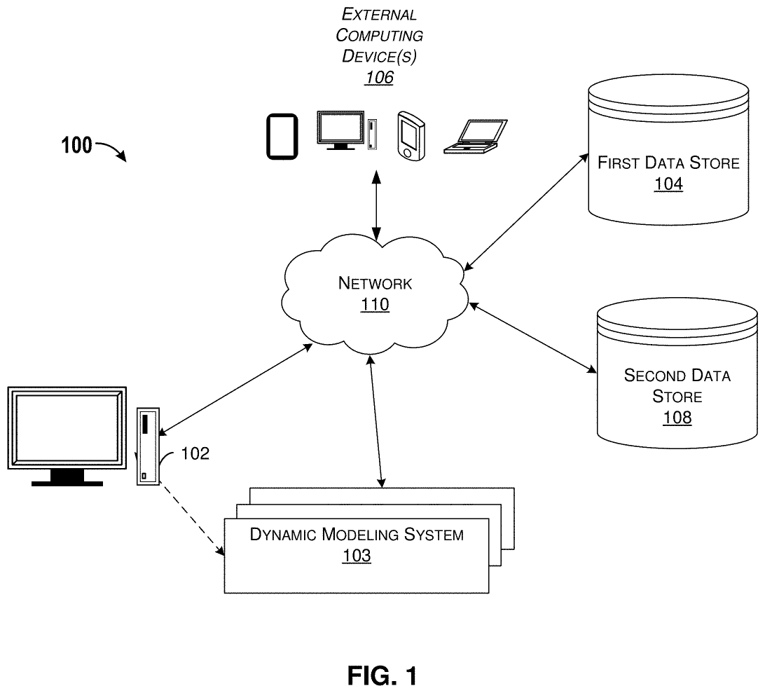

FIG. 1 shows a network diagram of various components that communicate via a communication network 110 and form an identity scoring platform 100 that provides scores for entities having records processed by the identity scoring platform 100. The platform 100 comprises a dynamic modeling system 103 interfacing with a computing device 102, a first data store 104, a second data store 108, and external computing devices 106, for example via a network 110. Additionally, communication links are shown enabling communication among the components of the platform 100 via the network 110.

The computing device 102 is shown communicatively coupled to the dynamic modeling system 103 via an optional localized manner (for example, via an optional local communication link) and in an external manner where communications occur through the network 110. In some embodiments, the dynamic modeling system 103 is integrated into the computing device 102 or vice versa. Furthermore, in some embodiments, one or more of the first data store 104 and the second data store 108 are combined into a single data store that is local to the computing device 102 or remote from the computing device 102 (not shown). In some embodiments, two or more of the components shown in FIG. 1 above are integrated, one or more components are excluded, or one or more components not shown in FIG. 1 are added to the platform 100. The platform 100 may be used to implement systems and methods described herein.

In some embodiments, the network 110 may comprise any wired or wireless communication network by which data and/or information may be communicated between multiple electronic and/or computing devices. The network 110 may be used to interconnect nearby devices or systems together, employing widely used networking protocols. The various aspects described herein may apply to any communication standard, such as a wireless 802.11 protocol. The computing device 102 may comprise any computing device configured to transmit and receive data and information via the network 110 for an entity. The entity may be a financial institution such as a business, bank, credit card company, a non-profit organization, an educational institution, a healthcare provider, an insurer, and so forth. In some embodiments, the computing device 102 may include or have access to one or more databases (for example, the first data store 104 and the second data store 108) that include various records and information that may be used to generate attributes and/or determinations between synthetic and real identities, and generate outputs corresponding thereto. These attributes (and corresponding information) may be used to dynamically generate models used to score identities and corresponding records when making the determinations between synthetic and real identities.

The computing devices 102, 106 may comprise any computing device configured to transmit and receive data and information via the network 110. In some embodiments, the computing device 102 represents a centralized computing device that performs at least a portion of the processing described herein. For example, the computing device 102 and/or dynamic modeling system 103 may implement the machine learning models described herein to generate scores for particular entities or identities and determine whether the particular entities or identities are real or synthetic. The computing device and/or dynamic modeling system may further analyze records stored in the first data store 104 and/or the second data store 108 and generate an output regarding the particular entities or identities and/or perform one or more actions based on the performed analysis and/or the transmitted and received data and information. In some embodiments, the computing devices 106 represent a customer device or user device that the customer or user utilizes to access the platform 100 and provide a target entity or identity that the platform 100 then determines to be either real or synthetic.

In some embodiments, the one or more computing devices 102, 106 comprise mobile or stationary computing devices. In some embodiments, the computing devices 106 provide users with remote access to the network 110 and the platform 100.

The first data store 104 may comprise one or more databases or data stores and may also store data regarding any identities (for example, name information, address information, activity information, and so forth). Using an example use case, the first data store 104 comprises credit data that includes name information, address information, contact information, financial information, as well as other credit related data. In some embodiments, the credit database may provide data for individuals or entities within particular geographic areas or for the entire platform 100.

The second data store 108 may also comprise one or more databases or data stores from a different source as compared to the first data store 104. The second data store 108 may also store data regarding or corresponding to entities or identities, for example relationship data, transaction data, behavioral data, and so forth. In the example use case, the second data store 108 comprises one or more of property rental or ownership information, membership data, marketing data, public records data, business information, eCommerce data, digital browsing or footprint data, location data, and/or other data. This data may be organized based on or according to identifiers common between the first data store 104 and the second data store 108, in some instances. In some embodiments, records associated with the same entity between different databases can be linked together using one or more methods described in U.S. provisional Application No. 62/900,341, filed Sep. 13, 2019, entitled “SINGLE IDENTIFIER PLATFORM FOR STORING ENTITY DATA,” which is incorporated by reference in its entirety herein. In some embodiments, one or more of the first data store 104 and the second data store 108 comprise data from publicly available and/or private sources.

The dynamic modeling system 103 may process data from the first data store 104 and the second data store 108 and also dynamically generate and/or apply one or more artificial intelligence models based on requests or inputs provided by users via the computing devices 106. The dynamic modeling system 103 may dynamically apply one or more machine learning models to data obtained from one or more of the first data store 104, the second data store 108, and/or the users (via the one or more computing devices 106). In some embodiments, the machine learning models may be generated or adapted dynamically by the dynamic modeling system 103 as the inputs and data change. For example, the dynamic modeling system 103 may generate changing machine learning (or other artificial intelligence) models in real-time based on the inputs received from the user (for example, a particular identifier, and so forth). In some embodiments, the generated models themselves may be dynamically applied to the inputs and data. For example, the models generated by the dynamic modeling system 103 may create various score, metrics, and/or data points based on the data sourced from the first and second data stores 104 and 108, respectively, and the users (for example, filters, threshold criteria, and so forth). In some embodiments, the dynamic modeling system 103 may automatically adjust the machine learning models to meet pre-selected levels of accuracy and/or efficiency.

In some embodiments, the dynamic modeling system 103 adapts to data from the first data store 104, the second data store 108, and/or from users that is constantly changing. For example, in one embodiment, the data in the first data store 104 and the second data store 108 is constantly updated and is different for each analysis of an identifier or entity. Using the example use case, a first user associated with a first financial institution may be interested in determining whether an identity (such as may be at least partially defined by personal information, such as a name, address, and SSN, provided to the first financial institution by a consumer purporting to be the person having the provided personal information) is real or synthetic using a first threshold set or amount of data (for example, in view of credit data only). A second user from a second financial institution may be interested in determining whether the identity is real or synthetic using all available information (including credit data, business data, property data, public records data, and so forth). Accordingly, the data obtained from the first and second data stores 104 and 108, respectively, using the criteria from the users, will likely be constantly changing. Thus, the processing and/or model generation performed by the platform 100 will change for each user. Additionally, the data obtained from the first and second data stores 104 and 108 will likely change over time as records in the data stores are updated, replaced, and/or deleted. Accordingly, the dynamic modeling system 103 may dynamically generate and/or apply machine learning models to handle constantly changing data and requests.

Based on details of the user requests, as will be detailed herein, the requests may be filtered to eliminate those requests that need not be completed. For example, in the example use case, requests may be filtered to eliminate those requests that are predetermined to involve real or synthetic identities or entities. Accordingly, the dynamic modeling system 103 may reduce unnecessary data processing by excluding certain requests.

In various embodiments, large amounts of data are automatically and dynamically processed interactively in response to user inputs, and the calculated data is efficiently and compactly presented to a user by the platform 100. Thus, in some embodiments, the data processing, machine learning, and generating of outputs and/or user interfaces described herein are more efficient as compared to previous data processing and output generation to identify false identities or entities.

The various machine learning models and processing of data to identify real and synthetic identities and entities, dynamic data processing, and output generation of the present disclosure are the result of significant research, development, improvement, iteration, and testing. This non-trivial development has resulted in the machine learning models and output generation described herein, which may provide significant efficiencies, improvements in accuracy and consistency, and advantages over previous systems.

Various embodiments of the present disclosure provide improvements to various technologies and technological fields. For example, as described above, existing data storage and processing technology (including, for example, in memory databases) is limited in various ways (for example, manual data review is slow, costly, and less detailed; data is too voluminous; and so forth), and various embodiments of the disclosure provide significant improvements over such technology. Additionally, various embodiments of the present disclosure are inextricably tied to computer technology. In particular, various embodiments rely on application of machine learning models, acquisition and processing of data, and presentation of output information via interactive graphical user interfaces or reports. Such features and others (for example, processing and analysis of large amounts of electronic data) are intimately tied to, and enabled by, computer technology, and would not exist except for computer technology. For example, the interactions with data sources and displayed data described below in reference to various embodiments cannot reasonably be performed by humans alone, without the computer technology upon which they are implemented. Further, the implementation of the various embodiments of the present disclosure via computer technology enables many of the advantages described herein, including more efficient interaction with, more accurate and consistent processing of, and presentation of, various types of electronic data.

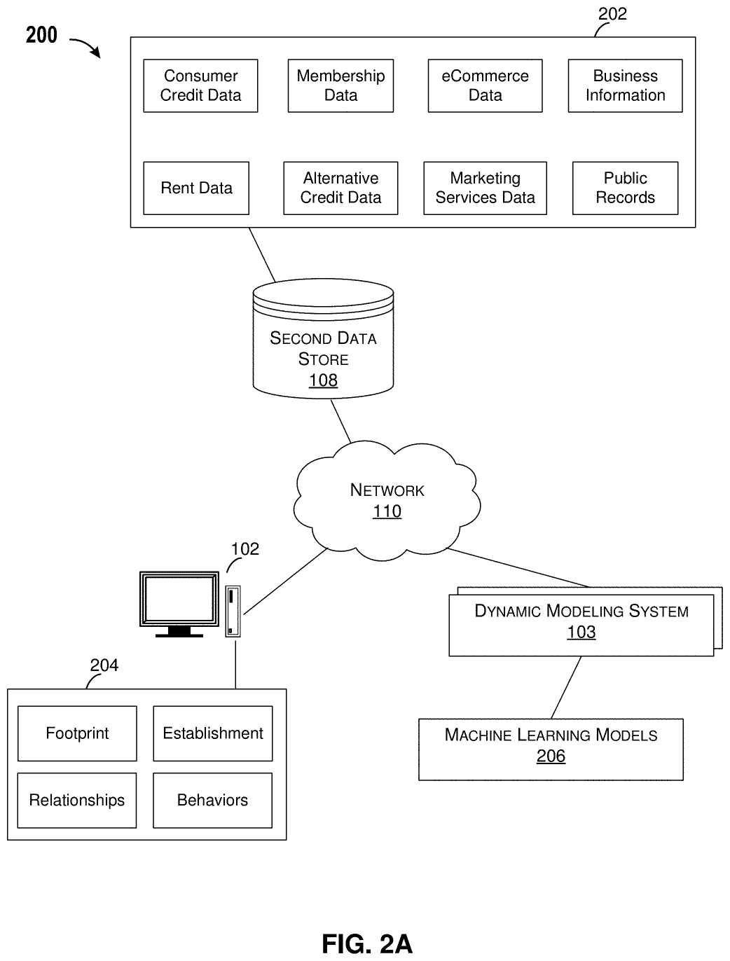

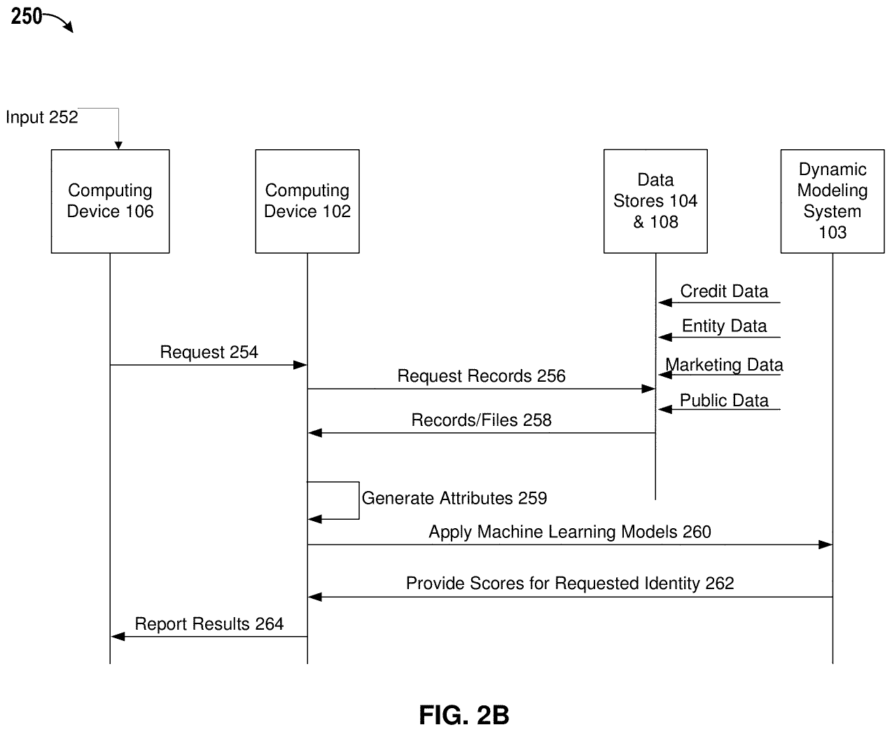

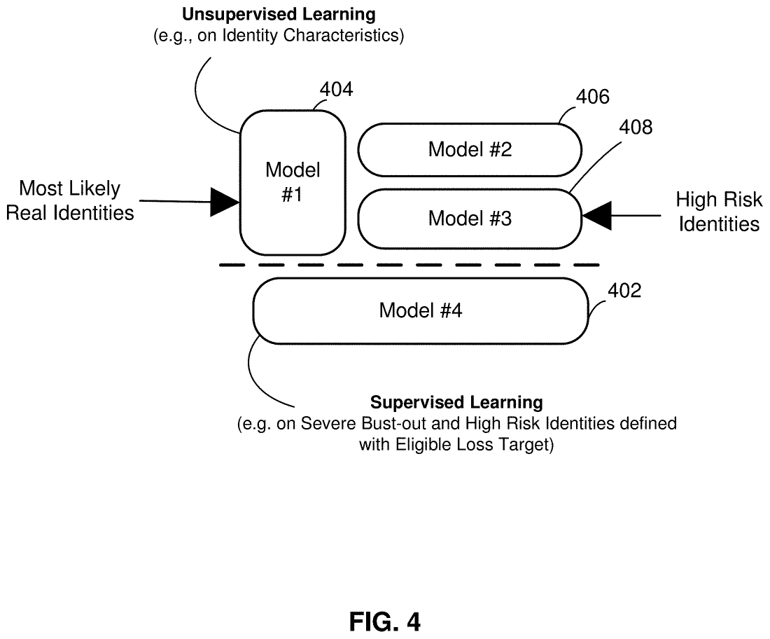

FIG. 2A shows a block diagram 200 indicating components within the identity scoring platform 100 of FIG. 1 that process data and apply machine learning models based on received requests. In FIG. 2A, data 202, in the form of records, etc., stored in the data stores 104 and 108 is received by the platform 100 via the data stores and the network 110. This data may come from a variety of selectable sources and may be aggregated over time. The platform 100 also utilizes attributes 204, for example provided by the computing device 102 via the network 110. As shown, in the illustrated embodiment of FIG. 2A, the attributes 204 for specific entities (such as specific identities of people) may include a digital footprint, an establishment age, one or more relationships (such as relationships to other entities), or one or more behaviors of the entity. In some embodiments, the platform 100 may generate the attributes based on the data 202 received from the data stores 104 and 108. For example, the data 202 dictates what relationships can be identified or what behaviors can be extracted from the data 202. The dynamic modelling system 103 may provide the platform 103 with various machine learning models 206. The platform 100 may use the models 206 to identify synthetic identities, as will be described in further detail herein, such as with respect to FIG. 4.

FIG. 2B shows a flow diagram 250 indicating data flow within the identity scoring platform of FIG. 1 based on a request to identify an identity score for a particular entity using a plurality of machine learning models. The flow diagram 250 begins with receiving an input 252 from a user or customer, for example requesting the platform 100 to identify whether a target identity is synthetic or real. The computing device 106 may receive the input 252 and may convey the input 252 as a request 254 to the computing device 102 via the network 110 (not shown in FIG. 2B). The request 254 may include the target identity. For example, the request may include portions of data provided by an applicant for credit purporting to be data about the applicant. Based on the received request 254, the computing device 102 may request corresponding records from the first and second data stores 104 and 108, respectively, at step 256. One or both of the data stores 104 and 108 may include various data (for example, credit data, entity data, marketing data, public data, and so forth). The first and second data stores 104 and 108 may provide the records to the computing device 102 at step 258. In some embodiments, providing the records to the computing device 102 comprises making the records available to the computing device 102 for retrieval. At 259, the computing device 102 may generate one or more attributes based on the records received from the first and second data stores 104 and 108. The attributes may be derived from the retrieved data based on predefined rule sets, functions, formulas, filters and/or models that may be applied to the data. For example, if the retrieved data includes a mailing address for an entity (e.g., a person or identity that is a subject of the request), an attribute derived based in part on the address and other retrieved records may be the number of other entities or identities that are also associated with the same address. Another attribute related to the same address data may be the amount of time (such as number of days) that the address has been associated with the entity (e.g., representing how long the person has lived at the given address).

At 260, the computing device 102 may apply one or more of the machine learning models using the dynamic modeling system 103. In some embodiments, applying the machine learning models comprises using one or more machine learning algorithms or models to the records received at 258, to the attributes generated at 259, and/or to the target identity. At 262, the dynamic modeling system 103 may return a score related to the target identity, which may be based on output of the machine learning models, where the score relates to whether or not the target identity is a synthetic identity or a real identity. At 264, the computing device 102 generates a report, output, notification, alert, or so forth for reporting to the user regarding the determination whether the target identity is a synthetic identity or real identity. For example, providing a notification to the user or an associated requesting entity may include generating and delivering (such as in a user interface, via an API response, and/or in another manner) notification that the identity is not a real identity (which may be due to determination that it is a synthetic identity). Alternatively, in at least some instances in which the identity is determined not to be synthetic, the notification may indicate that the identity is real, and may optionally include information (such as text, a graphical symbol, an API token, a field value, and/or other form of information) indicating that the identity will be guaranteed against at least a portion of any subsequent losses stemming from synthetic identity fraud, as discussed further herein.

The result may include or be used to generate a guarantee to a requesting user or entity that the person or entity that was the subject of the request 254 is a real identity, such that an operator of the dynamic modeling system 103 and/or computing device 102 will reimburse financial losses to the requesting user that can be tied to a showing that the subject entity was in fact using a synthetic identity as defined by the logic used in the first layer analytical framework to open an account (such as a credit account opened by the requesting financial on behalf of a synthetic identity). In such embodiments, the methods described herein (which may include providing identity data from a requesting entity as input to multiple machine learning models described below) may be performed in real time in response to an identity verification request, such that the most up to date database records are utilized by the models in generating a synthetic identity risk score and an optional guarantee against synthetic identity to the requesting entity (e.g., if the scores and/or aggregate score from the models meets a threshold and/or rule set that triggers a guarantee). In other embodiments, aspects of the synthetic identity detection described herein may be applied on a batch basis or periodically to flag records in a credit bureau and/or other database(s) that may be associated with a synthetic identity. In some embodiments, the platform may use the results to generate an output indicating that the target identity is synthetic and/or cannot be guaranteed.

In embodiments in which methods described herein are used by an operator of the system, such as a credit bureau or authentication service, to guarantee that a given identity is not synthetic, it is desirable to implement additional steps to identify an association between an inquiry (such as an identity authentication request or a credit inquiry) coming from a requesting entity (who may be the beneficiary of the guarantee), such as at the time of a credit application or credit inquiry, to later trade records (such as a credit tradeline) that are reported by the entity in subsequent months. For example, if a credit bureau will issue a guarantee to a financial institution that a particular identity submitted in a credit application to the financial institution is real and not a synthetic identity, it is desirable for the credit bureau to implement steps to link an account subsequently appearing on a credit file for that identity at that financial institution as the account for which financial losses may be subject to reimbursement by the credit bureau. There are at least two challenges that arise in attempting to implement a solution to this problem, discussed below.

One challenge for matching an inquiry and a subsequent tradeline in a credit file, for example, is that most of the fields populated in an inquiry may not be matching to the fields in the tradeline. For example, a credit inquiry may include fields such as a subscriber code, a kind of business (of the requesting entity), and an inquiry purpose type (such as indicating what permissible purpose the requesting entity has for a credit inquiry). These fields may not match or have counterparts in the trades opened following this inquiry. That may leave the system to rely on only a small subset of fields, such as identifiers of the consumer and the financial institution, to pull candidate inquiry-trade pairs, which usually leads to a large number of possible candidate pairs. As another challenge, not every inquiry leads to a corresponding trade or tradeline later appearing. For example, the credit inquiry may not result in an account opening (e.g., the financial institution could deny an account, such as due to a low credit score). Furthermore, a trade may not always trace back to an inquiry to the particular credit bureau or other system operator that processed the inquiry (e.g., the financial institution may have only sent the original credit inquiry to a different credit bureau).

In some embodiments, the system may match an inquiry to a subsequent trade (e.g., a subsequently opened account, as appearing a month or some other time after the original inquiry received a guarantee against synthetic identity-related financial loss) as a two-step process. First, the system may determine a pool of potential linkages between inquiries and trades (e.g., as inquiry-trade pairs). As an example, the condition to enter this pool may be (a) the inquiry and the trade have the same consumer identifier (e.g., a ConsumerID field value), (b) the trade is opened not earlier than the date of the inquiry, but not more than a predetermined number of days after the inquiry date, and (c) the inquiry and the trade are from the same client entity (e.g., they both include the same companyID value associated with the requesting financial institution).

Once the pool of inquiry-trade pairs is determined, this pool is still a many-to-many mapping. But according to the nature of a credit inquiry, the linkage should be one-to-one (one inquiry can only link to at most one trade and one trade only to at most one inquiry). The system may resolve this problem by effectively mapping the problem into a weighted maximum bipartite matching problem, which is a mathematical problem that has been applied in other domains. The inquiry-trade linkage pool is formed into a bipartite graph, where the graph is formed with two disjoint sets of vertices, with connections/edges only from a vertex in one set to a vertex in the other set, where inquiries and trades each form one of the independent vertex sets. The system then proceeds to assign edges between the two vertex sets so that: (a) the two vertices of each assigned edge satisfy one or more predefined requirements (in one embodiment, the condition mentioned above that the trade is opened not earlier than the date of the inquiry, but not more than a predetermined number of days after the inquiry date), (b) no two edges share one vertex, and (c) the total weight of the assigned edges is optimized. While there are several ways to approach this problem, trials of an approach utilizing the known “Hungarian algorithm” assigned the correct linkage in over 98% of cases during testing.

Difficulties training supervised machine learning based models may result in reduced capabilities of detecting synthetic identities and entities. Through extensive and iterative testing and examination of historical and behavioral aspects of records as they relate to identity behavior, the platform 100 identified and applies various characteristics for identifying synthetic identities. First, synthetic identities often use fragments of real information in order to fool financial institutions and gain access to credit or similar accounts. Second, synthetic identities often accumulate multiple credit trades and then rapidly “bust-out” by making large purchases and never repay the associated debts. Additionally, synthetic identities may have records that exist in a small number of sources or be linked to large numbers of straight rolled charge-offs. As a further difficulty, it may be difficult to define and identify what is in fact a synthetic identity even in hindsight, which may lead to limited historical training data for a supervised machine learning model or training data that does not include every type or pattern of synthetic identity.

The platform 100 can target these behaviors to better identify suspected synthetic identities or entities by focusing on the intersection of at least these characteristics. For example, logic establishing criteria for performing an analysis on a requesting identity can assist in determining whether the identity is real or synthetic. Such criteria may limit applying the machine learning models and additional processing described herein based on threshold information, for example the type of event in question (for example, request for a new line of credit, a new transaction, and so forth) or details of the entity or identity in question. In some embodiments, the logic determines whether a particular target identifier or a synthetic identifier includes suspicious identity behavior and abnormal bust out behavior. The logic may be used as criteria for determining reimbursement, as described herein.

When the thresholds are met, the platform can apply one or more machine learning models to help identify whether the target entity or identity in question is synthetic or real. The machine learning model applied can be selected from any number of available machine learning models. In some embodiments, the platform 100 applies multiple machine learning models to determine whether the target entity or identity is synthetic or real. For example, to identify synthetic identities, the available machine learning models are broadly divided into two categories based on whether or not a pseudo target is used for training. Thus, the machine learning models available to be applied by the platform 100 may utilized supervised learning or unsupervised learning approaches (which may broadly include what may be considered semi-supervised learning approaches).

As will be described herein, even though unsupervised learning approaches focus on identifying synthetic identities without training on a specific target, each still provides strong predictive power in separating the synthetic identities from real identities. Furthermore, each of the unsupervised learning approaches may provide information to one or more of the supervised learning approaches. In some embodiments, by combining all of the machine learning models (supervised and unsupervised learning approaches), detection of synthetic identities can be improved as compared to detection by individual models. An important benefit of the combined application of the machine learning models is detection of suspicious behaviors for requests that meet and/or do not meet the request criteria described herein. In some embodiments, individual scores from individual models may be combined with or merged with or otherwise supplement each other to provide a total or overall score for the target identity. If the target identity has an overall score that is greater than a specified threshold, then the platform 100 may identify the target identity as being synthetic. Alternatively, or additionally, the platform 100 may determine, based on these same scores, that the target identity is not synthetic and can be guaranteed. It will be appreciated that while a high score (e.g., at or above the threshold) in such an embodiment may represent confidence that an identity is synthetic, other embodiments may be implemented where a high score represents a confidence that an identity is real or authentic (e.g., a confidence that the identity is not synthetic).

In some embodiments, one or more of the machine learning models described herein heavily leverage identity and relation information from the first data store 104 and/or the second data store 108 for the purpose of detecting suspicious credit activities (indicative of a potential synthetic identity).

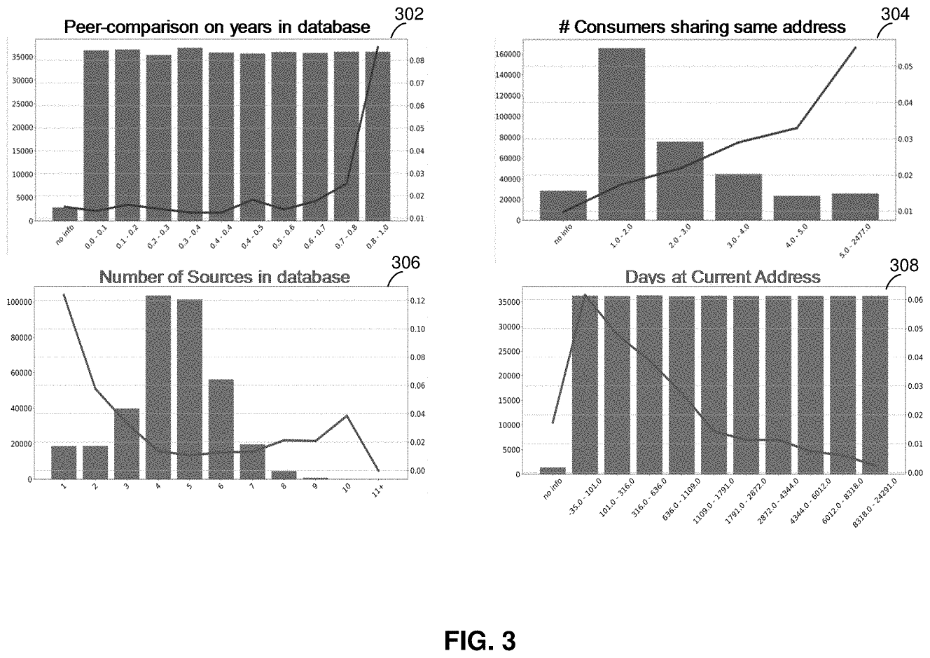

The data stores 104 and/or 108 may contain billions of records of identity information contributed to by various sources. For example, the records include consumer credit information, membership information, commerce/eCommerce information, business information, residential/housing information, alternative credit information, marketing services, public record information, and so forth. In some embodiments, this information includes digital contact information, location information, online historical information, data from/regarding mobile devices and interactions, and so forth. In view of this quantity of data, the platform 100 may utilize various attributes to identify and target different aspects of consumers' identities and non-credit behavior, for example along four major categories, for example attributes 204 as shown in FIG. 2A. These attributes may enable the platform 100 to effectively detect abnormal identity behavior (for example, often associated with synthetic identities) in a scalable manner for one or more of the machine learning models. FIG. 3 shows examples of some of these attributes.

FIG. 3 shows graphs 302, 304, 306, and 308 representing how a sample of attributes described herein correlate to a likelihood of being a synthetic identity or entity. Data for the bar graphs can be found along the left axis and the bottom axis of each graph, while data for the overlying lines can be found along the right axis and the bottom axis of each graph. The graph 302 shows how a peer comparison on years in a data source (for example, one of the first data store 104 and the second data store 108), as shown by the bar graphs, relate to an indicator or score (line overlaying the bar graphs) that may be indicative of a likelihood that a corresponding identity is synthetic (or shares features with a higher number of synthetic identities). The graph 304 shows how a number of consumers sharing a same address (bar graphs) relates to an indicator or score (line overlaying the bar graphs) that may be indicative of a likelihood that a corresponding identity is synthetic (or shares features with a higher number of synthetic identities).

For example, an address that is shared by 10 or more entities has a higher likelihood of being associated with a synthetic identity as compared to an address associated with only one entity. The graph 306 shows how a number of sources in which data relating to a record or identity is found (bar graphs) relates to an indicator or score (line overlaying the bar graphs) that may be indicative of a likelihood that a corresponding identity is synthetic (or shares features with a higher number of synthetic identities). For example, a record or entity having data stored in only one source has a higher likelihood of being associated with a synthetic identity as compared to a record or entity having data stored in five sources. The graph 308 shows how a number a number of days at which an entity has been at a current address (bar graphs) relates to an indicator or score (line overlaying the bar graphs) that may be indicative of a likelihood that a corresponding identity is synthetic (or shares features with a higher number of synthetic identities). For example, an entity having less than 101 days at the current address has a higher likelihood of being associated with a synthetic identity as compared to an entity having greater than 8000 days at the current address. In some embodiments, these attributes may result in the corresponding records, entities, or identities having scores or values associated therewith that are used to determine whether an identity or entity is synthetic. While some sample attributes are shown, there may be a large number of attributes in some embodiments (such as over 100 attributes) that may be provided as features or input to one or more of the models.

FIG. 4 shows an example of machine learning models used by the platform of FIG. 1. The machine learning models include a first supervised machine learning model 402, a first unsupervised machine learning model 404, a second semi-supervised machine learning model 406, and a third unsupervised machine learning model 408. As described herein, the model 402 may be a supervised learning model while the models 404, 406, and 408 use unsupervised learning. Additionally, the models may use real identities along with synthetic or high risk identities, as described herein.

The first supervised machine learning model 402 may comprise a target model that uses the attributes described herein to generally identify records that could be associated with synthetic identities as opposed to real identities based on comparisons to known relationships (for example, labeled records). The first supervised model may monitor high risk identities (for example, identities likely to be synthetic) and identities with severe bust outs or large numbers of chargeoffs as compared to a known target. The first supervised machine-learning model may use the attributes to determine scores for the target identity from the received request based on how the target identity compares to other records relative to the attributes. In some embodiments, the first supervised machine-learning model may use the logic of the first layer of analytical framework as a pseudo-target and train. In some embodiments, the first supervised machine learning model may use a loss value of the target defined by the logic of the first layer of analytical framework as a pseudo-target and train. In some embodiments, this model uses a gradient boosting machine model with binned attributes and with monotonic constraints applied on certain attributes.

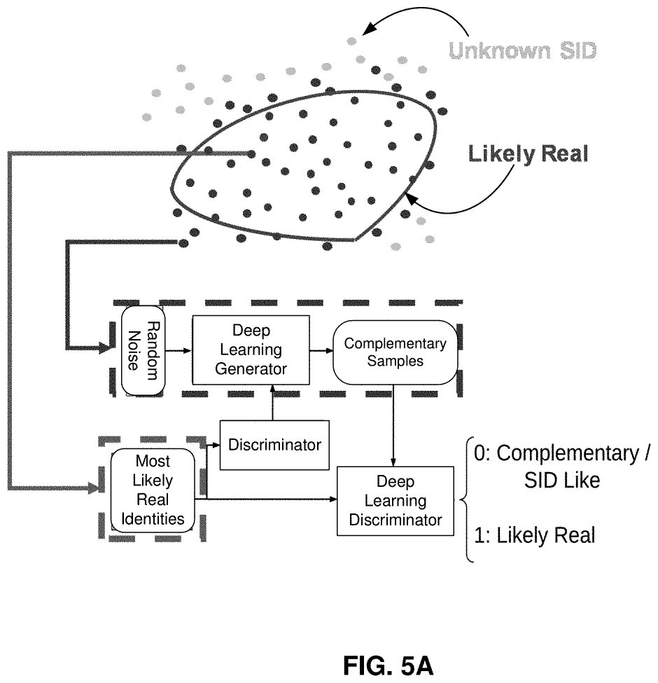

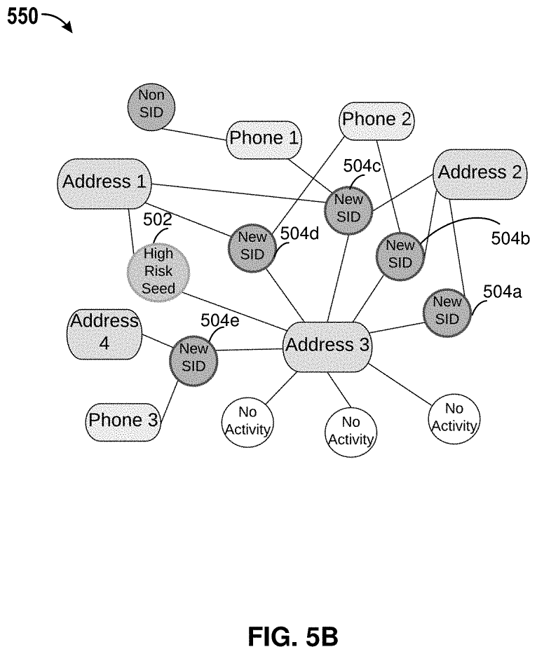

The first, unsupervised machine learning model 404 used to identify synthetic identities may be a one-class adversarial nets (OCAN) machine learning model. The OCAN model may rely on that fact that since synthetic identities are fabricated (often using real information), the synthetic identities may have characteristics and behaviors (for example, based on the information forming the synthetic identities) that differentiate the synthetic identities from real identities. The OCAN model utilizes a classifier (learned based on the data provided to the model) that distinguishes the synthetic identities from real identities. The OCAN model may be a type of generative adversarial network (GAN) which can be used to faithfully learn the distribution of data. The OCAN model extends the features of the GAN to train a discriminator of a complementary GAN model that is different from a general GAN model. For example, the OCAN model contains two phases during training. The first phase comprises learning user representations, or the real identities. Once the real identities are learned, the second phase is to train a complementary GAN to generate complementary samples that are in a “low-density” area of normal identities. The second phase further involves training a discriminator that can clearly distinguish the real identities from the complementary, or synthetic identities. Thus, the OCAN model may learn to discriminate synthetic identities based on learning about most likely real identities. A sample OCAN model is shown in FIG. 5A, which will be described further below. The OCAN may use a population most likely to be real identities to learn a complementary population that is closer to synthetic identities and uses a distinguisher to classify the complementary population (e.g., the synthetic identities) from the real identities.

The OCAN model used by the platform 100 may be learned and/or trained based on real identities using corresponding credit behavior and richness of identity information. The OCAN model may generate scores for real identities and synthetic identities. The results provided by the OCAN model may show a clear score separation between the real identities and synthetic identities, thereby showing the effectiveness of the OCAN model in identifying real versus synthetic identities. Thus, the scores generated by the OCAN model can be used as an orthogonal identity score. Such a use may complement scores generated by supervised models (for example the supervised target model) for synthetic identity detection.

A second, semi-supervised machine learning model used to identify synthetic identities may learn from or by generalizing suspected synthetic (or high likelihood or risk) identities. The second model may be a semi-unsupervised Deep Generative Model (SU-DGM). Semi-supervised learning may be used when a target within a dataset is sparsely labelled. In the case of synthetic identity detection, a (albeit limited or small) number of suspected synthetic (or high risk) identities in the data from the data stores 104 and 108 allow the platform 100 to accurately and consistently identify synthetic identities. Known synthetic identities may be identified or reported by financial institutions when they confirm fraud occurs related to a particular identity. However, false positives (for example, identities that are suspected as being synthetic but not confirmed as such) may not be verified or checked. As such, the platform 100 may only be aware of which identities are confirmed to be synthetic but may not receive feedback regarding suspected synthetic identities. In other words, there may not be a target label for such suspected synthetic identities. Furthermore, there may be multiple types of synthetic identities and some of them may be entirely unobserved in the labelled population of the data from the data stores 104 and 106. Therefore, semi-unsupervised learning may be used to identify synthetic identities accurately and consistently. The SU-DGM may generalize synthetic identity characteristics from a small population that scored highly for one specific type of synthetic identity fraud and identity other identities that are synthetic based thereon.

Equation 1 below comprises a loss function for the case the label/target is available. Equation 2 below comprises a second loss function for the case where the label/target is not observed. In some embodiments, the SU-DGM, may use a Gaussian mixture deep generative model to learn latent vector characteristics of the data of interest (for example, of the synthetic identities). In a deep generative model, parameters of distributions within a probabilistic graphical model are themselves parameterized by neural networks. In the SU-DGM, an inference model (e.g., an encoder) may learn from label information, when available, and the latent spaces spanned by a set of conditional Gaussian latent variables on the input identity data and predicted labels/available labels. Then, a generative model (e.g., a decoder) may attempt to reconstruct input identity data, with training guided by a combination of Equation 1 and Equation 2.

$$\begin{matrix}ℒ ( x , y ) = - {KL}_{z} ( {q}_{ϕ} ( z \vert x , y ) ‖ {p}_{θ} ( x , y , z ) ) & ( 1 ) \\ ℒ ( x ) = - {KL}_{z , y} ( {q}_{ϕ} ( z , y \vert x ) ‖ {p}_{θ} ( x , y , z ) ) & ( 2 )\end{matrix}$$

Where:
x is the input variables,
y is the limited label when available,
z is the latent variables,
q is the encoder,
p is the decoder, and
KL measures the divergence of the two distributions p and q

The SU-DGM may leverage knowledge regarding the synthetic identities (for example, the riskiest population in the data) having a high score for at least one type of synthetic identity (for example, authorized user fraud) having “labels” in the training data. The SU-DGM then generalizes to other suspicious synthetic identities that share the same latent space but that may display different abnormalities (or differences from real identities). Score distributions generated by this SU-DGM show a clear score separation between the real identities and synthetic (or risky) identities. Thus, the scores generated by the SU-DGM can be used as an orthogonal identity score. Such a use may complement scores generated by supervised models for synthetic identity detection.

A third model used to identify synthetic identities may also use suspected synthetic (or high risk) identities, similar to the SU-DGM. The third model may comprise a risk propagation graph score (RPGS) model. The RPGS model may identify records that are “related” to the suspected synthetic (or high risk) identities. The RPGS may assume that for an entity that utilizes organized (or multiple) synthetic identities, the multiple synthetic identities likely share some information with each other (for example, contact information, name information, and so forth) and likely are not all created or harvested at the same time. Thus, the more closely related one pin record (e.g., target identity) is to a seed population (either a confirmed synthetic identity or a suspected synthetic (or high risk) identity), the more likely the pin record is also a synthetic identity. The RPGS model may identify organized fraud rings by propagating a risk or likelihood of fraud based on fraudulent users that share identity information.

In some embodiments, the “closeness” of this relation may comprise three aspects: (1) how many data points (for example, trade, address, phone number, social security numbers (SSNs)) the two pins (for example, the target pin record and the confirmed or suspected synthetic identity) share with each other and how long the information has been shared; (2) how many pins share the same data points in a same path as the two pins; and (3) how many seeds the target pin is related to.

In databases including billions of records, the platform 100 may be challenged by many registered shared data points (for example, address, phone number, and SSN) that are not representative of meaningful connections. As such, the platform 100 may utilize filters to exclude identity elements which create thousands (and sometimes millions) of links from the data stores 104 and 108, which otherwise would easily skew identification of synthetic identities. Examples of filters that exclude irrelevant relationships (for example, noise) in the data include: (1) filters that exclude business addresses (for example, unit types being “STE” or “SUITE”); (2) filters that exclude high-rise building addresses where no unit number provided; and (3) filters that exclude SSNs and phone numbers with fewer than 3 distinct numbers (for example: 787778778).

The RPGS model may use a graph connecting the identities using the aforementioned relations and propagate an associated synthetic identity risk or score associated with the seed population along connections to neighboring pins and further to neighbors of the neighboring pins iteratively with attenuation. Weights on the 0^{th} layer of such a graph (an example of which is shown in FIG. 5B) may defined by Equation 3.

wᵢᵢ⁽⁰⁾=F(Pᵢ) (3)

Where: P₁ is the probability that identity i is a high risk or synthetic identity and α is an adjustable hyper parameter. Then, the synthetic identity risk or scores are propagated through the edges in the graph, with another hyper parameter b, where the weights of the non-O^{th} layers may be defined by Equation 4.

wᵢⱼ^{(l)}=A₁[{Gᵢₖ(wᵢₖ^{(l-1)},fₖⱼ))|∀k∈Lᵢ^{(l-1)}}] (4)

A synthetic identity or risk score of any one pin j may be generated based on all seeds according to Equation 5.

ρᵢ=A₂[{A₃({wᵢⱼ^{(l)}|∀l})|∀i∈S}] (5)

Where:
S={s₁, s₂, . . . sₙ} are the set of high risk identity seeds
Pᵢ is the known risk of the high-risk identity seeds.
wᵢⱼ^{(l)} is the weight we assign to pin j in the lth layer of hop from seed i
fᵢⱼ is a list of some properties of the identifier elements (address/phone/SSN/trade) that connects pin i and pin j. fᵢⱼ can be, but is not necessarily limited to:
   The number of pins that share this identifier elements
   When the identifier element is an address, whether it is an apartment building/townhouse/single family house/commercial address/etc.
   When the identifier element is a phone number, whether it is likely a fake phone number or not, for example (7474747474)
   When the identifier element is a last name, whether it is a commonly used first name, so the connection is caused by possible data entry error.
Lᵢ^{(l)} is the set of pins that are 1 hops away from seed i
ρⱼ is the final risk RPGS model assign to pin j.
F, G are some transformation functions, they can be, but are not necessarily limited to:

F(x)=\sqrt{x}

G(x,f)=x²/f
A₁, A₂, A₃ are some aggregation functions, they can be, but are not necessarily limited to:
   Mean
   Sum
   Max
   Mean square root

The calculation can be carried out for a limited number of layers (l<lₘₐₓ). If the functions (F, G, A₁, A₂, A₃) are designed properly and the computation power allows, the calculation can be carried out for unlimited number of layers and terminate once a convergence is reached.

The graph 550 shown in FIG. 5B comprises a detected organized ring of synthetic identities. By propagating from high risk seeds (for example, identities or records highly likely to be synthetic) in circle 502, the RPGS model may identify highly concentrated group of potential synthetic identities in circles 504a-504e (each identified as a New Synthetic Identity (SID) in FIG. 5B) that have trades opened subsequently to the high risk seed circle 502. The RPGS model is able to identify that these synthetic identities share various identity elements, such as addresses and phone numbers, among them.

Using these identified synthetic identities or scores, the platform 100 may generate an output to a received request regarding the target identity that identifies whether that target identity is synthetic or real. Thus, based on the machine learning models described herein and the generated scores, the platform effectively reduces risk by identifying the synthetic identities accurate and consistently. Thus, the platform 100, implementing the models described herein improve over existing methods and systems that are unable to differentiate synthetic identities from real identities well.

FIG. 5A is a block diagram of a first example machine learning model applied to the records to identity a first identity score. As shown in FIG. 5A, the OCAN model uses various computing blocks and values to identify the likely real identities and the likely synthetic identities by training discriminators. The OCAN may be trained based on a hypothesis that there is a population of known real identities, and that others are synthetic. A discriminator is trained to identify whether provided identity information appears to be a real identity. The training data may include, for example, records known to be associated with a real identity. These real identities may include, in the credit bureau context, both real people with good credit as well as real people with negative credit (e.g., an individual who defaulted on loans, declared bankruptcy, but later recovered from bankruptcy). The discriminator may be trained to approximate real identities that are on the border of real and synthetic (e.g., those just outside or near the “likely real” boundary in FIG. 5A). These borderline cases may be generated by the generator (such as from real identities populated with noise or some randomness). The result of training the discriminator on these borderline cases will typically also result in the discriminator detecting the synthetic identities falling further outside of the “likely real” boundary of FIG. 5A.

FIG. 5B is a block diagram of a second example machine learning model applied to the records to identity a second identity score. FIG. 5B shows how particular records can be determined to be synthetic by their relations with a known or high risk synthetic identity. FIG. 5B has been described in more detail above.

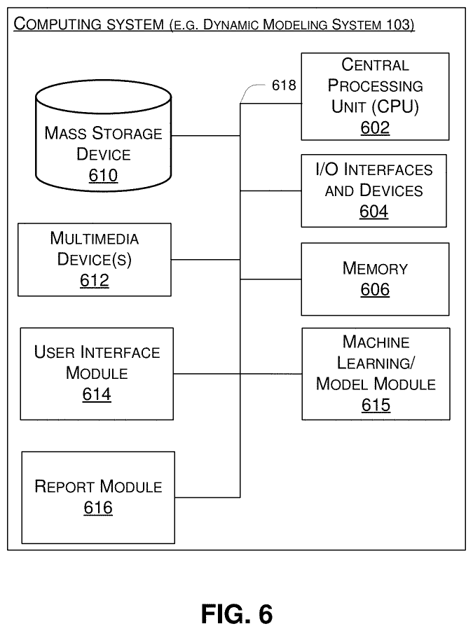

FIG. 6 is a block diagram showing example components of the identity scoring platform of FIG. 1.

The hardware and/or software components, as discussed below with reference to the dynamic modeling system 103 may be also included in any of the devices of the platform 100 (for example, the computing device 102, the computing devices 106, and so forth). These various depicted components may be used to implement the systems and methods described herein.

In some embodiments, certain modules described below, such as the modeling module 615, a user interface module 614, or a report module 616 included with the dynamic modeling system 103 may be included with, performed by, or distributed among different and/or multiple devices of the platform 100. For example, certain user interface functionality described herein may be performed by the user interface module 614 of various devices such as the computing device 102 and/or the one or more computing devices 106.

In some embodiments, the various modules described herein may be implemented by either hardware or software. In an embodiment, various software modules included in the dynamic modeling system 103 may be stored on a component of the dynamic modeling system 103 itself (for example, a local memory 606 or a mass storage device 610), or on computer readable storage media or other component separate from the dynamic modeling system 103 and in communication with the dynamic modeling system 103 via the network 110 or other appropriate means.

The dynamic modeling system 103 may comprise, for example, a computer that is IBM, Macintosh, or Linux/Unix compatible or a server or workstation or a mobile computing device operating on any corresponding operating system. In some embodiments, the dynamic modeling system 103 interfaces with a smart phone, a personal digital assistant, a kiosk, a tablet, a smart watch, a car console, or a media player. In some embodiments, the dynamic modeling system 103 may comprise more than one of these devices. In some embodiments, the dynamic modeling system 103 includes one or more central processing units (“CPUs” or processors) 602, 110 interfaces and devices 604, memory 606, the machine learning model module 615, a mass storage device 610, a multimedia device 612, the user interface module 614, a report module 616, and a bus 618.

The CPU 602 may control operation of the dynamic modeling system 103. The CPU 602 may also be referred to as a processor. The processor 602 may comprise or be a component of a processing system implemented with one or more processors. The one or more processors may be implemented with any combination of general-purpose microprocessors, microcontrollers, digital signal processors (“DSPs”), field programmable gate array (“FPGAs”), programmable logic devices (“PLDs”), controllers, state machines, gated logic, discrete hardware components, dedicated hardware finite state machines, or any other suitable entities that can perform calculations or other manipulations of information.

The I/O interface 604 may comprise a keypad, a microphone, a touchpad, a speaker, and/or a display, or any other commonly available input/output (“I/O”) devices and interfaces. The I/O interface 604 may include any element or component that conveys information to the user of the dynamic modeling system 103 (for example, a requesting dealer, manufacturer, or other entity) and/or receives input from the user. In one embodiment, the I/O interface 604 includes one or more display devices, such as a monitor, that allows the visual presentation of data to the consumer. More particularly, the display device provides for the presentation of GUIs, application software data, websites, web apps, and multimedia presentations, for example.

In some embodiments, the I/O interface 604 may provide a communication interface to various external devices. For example, the dynamic modeling system 103 is electronically coupled to the network 110 (FIG. 1), which comprises one or more of a LAN, WAN, and/or the Internet. Accordingly, the I/O interface 604 includes an interface allowing for communication with the network 110, for example, via a wired communication port, a wireless communication port, or combination thereof. The network 110 may allow various computing devices and/or other electronic devices to communicate with each other via wired or wireless communication links.

The memory 606, which includes one or both of read-only memory (ROM) and random access memory (“RAM”), may provide instructions and data to the processor 602. For example, data received via inputs received by one or more components of the dynamic modeling system 103 may be stored in the memory 606. A portion of the memory 606 may also include non-volatile random access memory (“NVRAM”). The processor 602 typically performs logical and arithmetic operations based on program instructions stored within the memory 606. The instructions in the memory 606 may be executable to implement the methods described herein. In some embodiments, the memory 606 may be configured as a database and may store information that is received via the user interface module 614 or the I/O interfaces and devices 604.

The dynamic modeling system 103 may also include the mass storage device 610 for storing software or information (for example, the generated models or data obtained to which the models are applied, and so forth. Software shall be construed broadly to mean any type of instructions, whether referred to as software, firmware, middleware, microcode, hardware description language, or otherwise. Instructions may include code (for example, in source code format, binary code format, executable code format, or any other suitable format of code). The instructions, when executed by the one or more processors, cause the processing system to perform the various functions described herein. Accordingly, the dynamic modeling system 103 may include, for example, hardware, firmware, and software, or any combination therein. The mass storage device 610 may comprise a hard drive, diskette, solid state drive, or optical media storage device. In some embodiments, the mass storage device may be structured such that the data stored therein is easily manipulated and parsed.

As shown in FIG. 2A, the dynamic modeling system 103 includes the modeling module 615. As described herein, the modeling module 615 may dynamically generate (such as through a training process) one or more models for processing data obtained from the data stores or the user. In some embodiments, the modeling module 615 may also apply the generated models to the data. In some embodiments, the one or more models may be stored in the mass storage device 610 or the memory 606. In some embodiments, the modeling module 615 may be stored in the mass storage device 610 or the memory 606 as executable software code that is executed by the processor 602. This, and other modules in the dynamic modeling system 103, may include components, such as hardware and/or software components, object-oriented software components, class components and task components, processes, functions, attributes, procedures, subroutines, segments of program code, drivers, firmware, microcode, circuitry, data, databases, data structures, tables, arrays, and variables. In the embodiment shown in FIG. 3, the dynamic modeling system 103 is configured to execute the modeling module 615 to perform the various methods and/or processes as described herein.

In some embodiments, the report module 616 may be configured to generate a report, notification, or output mentioned and further described herein. In some embodiments, the report module 616 may utilize information received from the dynamic modeling system 103, the data acquired from the data stores, and/or the computing device 102 or the user of the computing device 106 or the computing device 102 of FIG. 2 to generate the report, notification, or output for a specific dealer, manufacturer, or other entity. For example, the dynamic modeling system 103 may receive information that the dealer, manufacturer, or entity provides via the network 110 that the dynamic modeling system 103 uses to acquire information from the data stores and generate models for processing of the information.

The dynamic modeling system 103 also includes the user interface module 614. In some embodiments, the user interface module 614 may also be stored in the mass storage device 610 as executable software code that is executed by the processor 602. In some embodiments, the dynamic modeling system 103 may be configured to execute the user interface module 614 to perform the various methods and/or processes as described herein.

The user interface module 614 may be configured to generate and/or operate user interfaces of various types. In some embodiments, the user interface module 614 constructs pages, applications or displays to be displayed in a web browser or computer/mobile application. In some embodiments, the user interface module 614 may provide an application or similar module for download and operation on the computing device 102 and/or the computing devices 106, through which the user may interface with the dynamic modeling system 103 to obtain the desired report or output. The pages or displays may, in some embodiments, be specific to a type of device, such as a mobile device or a desktop web browser, to maximize usability for the particular device. In some embodiments, the user interface module 614 may also interact with a client-side application, such as a mobile phone application, a standalone desktop application, or user communication accounts (for example, e-mail, SMS messaging, and so forth) and provide data thereto.

For example, as described herein, the dynamic modeling system 103 may be accessible to the financial institution via a website or application programming interface (API).

Once the dynamic modeling system 103 receives the inputs or a request, a user or entity may view information or results via the I/O interfaces and devices 604 and/or the user interface module 614, in some embodiments. Once the dynamic modeling system 103 receives the information from the data stores (for example, via the I/O interfaces and devices 604 or via the user interface module 614), the processor 602 or the modeling module 615 may store the received inputs and information in the memory 606 and/or the mass storage device 610. In some embodiments, the received information from the data stores may be parsed and/or manipulated by the processor 602 or the dynamic modeling system 103 (for example, filtered or similarly processed).

In some embodiments, the processor 602 or the modules 616 or 617, for example, may be configured to generate ratings or levels (for example, a numerical rating or level) for models generated by the modeling module 615. In some embodiments, the ratings or levels may correspond to a confidence level in the accuracy of the modeling or other data processing. For example, the rating or level may provide a relative ranking of a specific model or data versus other models or data. In some embodiments, the rating or level may provide an absolute rating or level. In some embodiments, when a rating or level of a model or data is higher than that of other models or data, the model or data with the higher rating has a higher confidence of being accurate.

The various components of the dynamic modeling system 103 may be coupled together by a bus system 618. The bus system 618 may include a data bus, for example, as well as a power bus, a control signal bus, and a status signal bus in addition to the data bus. In different embodiments, the bus could be implemented in Peripheral Component Interconnect (“PCI”), Microchannel, Small Computer System Interface (“SCSI”), Industrial Standard Architecture (“ISA”) and Extended ISA (“EISA”) architectures, for example. In addition, the functionality provided for in the components and modules of the dynamic modeling system 103 may be combined into fewer components and modules or further separated into additional components and modules than that shown in FIG. 6.

### Computing Systems

Any of the components or systems described herein may be controlled by operating system software, such as Windows XP, Windows Vista, Windows 7, Windows 8, Windows Server, UNIX, Linux, SunOS, Solaris, iOS, Android, Blackberry OS, or other similar operating systems. In Macintosh systems, the operating system may be any available operating system, such as MAC OS X. In other embodiments, the components or systems described herein may be controlled by a proprietary operating system. Conventional operating systems control and schedule computer processes for execution, perform memory management, provide file system, networking, I/O services, and provide a user interface, such as a graphical user interface (“GUI”), among other things.

Computing devices, which may comprise the software and/or hardware described above, may be an end user computing device that comprises one or more processors able to execute programmatic instructions. Examples of such computing devices are a desktop computer workstation, a smart phone such as an Apple iPhone or an Android phone, a computer laptop, a tablet PC such as an iPad, Kindle, or Android tablet, a video game console, or any other device of a similar nature. In some embodiments, the computing devices may comprise a touch screen that allows a user to communicate input to the device using their finger(s) or a stylus on a display screen.

In general, the word “module,” as used herein, refers to logic embodied in hardware or firmware, or to a collection of software instructions, possibly having entry and exit points, written in a programming language, such as, for example, Java, Lua, C or C++. A software module may be compiled and linked into an executable program, installed in a dynamic link library, or may be written in an interpreted programming language such as, for example, BASIC, Perl, or Python. It will be appreciated that software modules may be callable from other modules or from themselves, and/or may be invoked in response to detected events or interrupts. Software modules configured for execution on computing devices may be provided on a computer readable medium, such as a compact disc, digital video disc, flash drive, or any other tangible medium. Such software code may be stored, partially or fully, on a memory device of the executing computing device, such as the platform 100 or the computing devices 102 and/or 106, for execution by the computing device. Software instructions may be embedded in firmware, such as an EPROM. It will be further appreciated that hardware modules may be comprised of connected logic units, such as gates and flip-flops, and/or may be comprised of programmable units, such as programmable gate arrays or processors. The modules described herein are in many embodiments implemented as software modules, but may be represented in hardware or firmware. Generally, the modules described herein refer to logical modules that may be combined with other modules or divided into sub-modules despite their physical organization or storage.

Each of the processes, methods, and algorithms described in the preceding sections may be embodied in, and fully or partially automated by, code modules executed by one or more computer systems or computer processors comprising computer hardware. The code modules may be stored on any type of non-transitory computer-readable medium or computer storage device, such as hard drives, solid state memory, optical disc, and/or the like. The systems and modules may also be transmitted as generated data signals (for example, as part of a carrier wave or other analog or digital propagated signal) on a variety of computer-readable transmission mediums, including wireless-based and wired/cable-based mediums, and may take a variety of forms (for example, as part of a single or multiplexed analog signal, or as multiple discrete digital packets or frames). The processes and algorithms may be implemented partially or wholly in application-specific circuitry. The results of the disclosed processes and process blocks may be stored, persistently or otherwise, in any type of non-transitory computer storage such as, for example, volatile or non-volatile storage.

The various features and processes described above may be used independently of one another, or may be combined in various ways. All possible combinations and sub combinations are intended to fall within the scope of this disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed example embodiments. The example systems and components described herein may be configured differently than described. For example, elements may be added to, removed from, or rearranged compared to the disclosed example embodiments.

Any process descriptions, elements, or blocks in the flow diagrams described herein and/or depicted in the attached figures should be understood as potentially representing modules, segments, or portions of code which include one or more executable instructions for implementing specific logical functions or blocks in the process. Alternate implementations are included within the scope of the embodiments described herein in which elements or functions may be deleted, executed out of order from that shown or discussed, including substantially concurrently or in reverse order, depending on the functionality involved, as would be understood by those skilled in the art.

All of the methods and processes described above may be embodied in, and partially or fully automated via, software code modules executed by one or more general purpose computers. For example, the methods described herein may be performed by the platform 100, a computing device 102 and/or 106, and/or any other suitable computing device. The methods may be executed on the computing devices in response to execution of software instructions or other executable code read from a tangible computer readable medium. A tangible computer readable medium is a data storage device that can store data that is readable by a computer system. Examples of computer readable mediums include read-only memory, random-access memory, other volatile or non-volatile memory devices, CD-ROMs, magnetic tape, flash drives, and optical data storage devices.

It should be emphasized that many variations and modifications may be made to the above-described embodiments, the elements of which are to be understood as being among other acceptable examples. All such modifications and variations are intended to be included herein within the scope of this disclosure. The foregoing description details certain embodiments of the disclosure. It will be appreciated, however, that no matter how detailed the foregoing appears in text, the disclosure can be practiced in many ways. As is also stated above, it should be noted that the use of particular terminology when describing certain features or aspects of the disclosure should not be taken to imply that the terminology is being re-defined herein to be restricted to including any specific characteristics of the features or aspects of the disclosure with which that terminology is associated. The scope of the disclosure should therefore be construed in accordance with the appended claims and any equivalents thereof.

The I/O devices and interfaces provide a communication interface to various external devices and systems. The computing system may be electronically coupled to a network, which comprises one or more of a LAN, WAN, the Internet, or cloud computing networks, for example, via a wired, wireless, or combination of wired and wireless, communication links. The network communicates with various systems or other systems via wired or wireless communication links, as well as various data sources.

The data sources 104 and 108 described herein may include one or more internal or external data sources. In some embodiments, one or more of the databases or data sources may be implemented using an open-source cross-platform document-oriented database program, such as a Mongo dB, a relational database, such as IBM DB2, Sybase, Oracle, CodeBase and Microsoft® SQL Server as well as other types of databases such as, for example, a flat file database, an entity-relationship database, and object-oriented database, and/or a record-based database.

It is recognized that the term “remote” may include systems, data, objects, devices, components, or modules not stored locally, that are not accessible via the local bus. Thus, remote data may include a system that is physically stored in the same room and connected to the computing system via a network. In other situations, a remote device may also be located in a separate geographic area, such as, for example, in a different location, country, and so forth.

### Additional Embodiments

It is to be understood that not necessarily all objects or advantages may be achieved in accordance with any particular embodiment described herein. Thus, for example, those skilled in the art will recognize that certain embodiments may be configured to operate in a manner that achieves or optimizes one advantage or group of advantages as taught herein without necessarily achieving other objects or advantages as may be taught or suggested herein.

All of the processes described herein may be embodied in, and fully automated via, software code modules executed by a computing system that includes one or more general purpose computers or processors. The code modules may be stored in any type of non-transitory computer-readable medium or other computer storage device. Some or all the methods may alternatively be embodied in specialized computer hardware. In addition, the components referred to herein may be implemented in hardware, software, firmware or a combination thereof.

Many other variations than those described herein will be apparent from this disclosure. For example, depending on the embodiment, certain acts, events, or functions of any of the algorithms described herein can be performed in a different sequence, can be added, merged, or left out altogether (for example, not all described acts or events are necessary for the practice of the algorithms). Moreover, in certain embodiments, acts or events can be performed concurrently, for example, through multi-threaded processing, interrupt processing, or multiple processors or processor cores or on other parallel architectures, rather than sequentially. In addition, different tasks or processes can be performed by different machines and/or computing systems that can function together.

The various illustrative logical blocks, modules, and algorithm elements described in connection with the embodiments disclosed herein can be implemented as electronic hardware, computer software, or combinations of both. To clearly illustrate this interchangeability of hardware and software, various illustrative components, blocks, modules, and elements have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various illustrative logical blocks and modules described in connection with the embodiments disclosed herein can be implemented or performed by a machine, such as a general purpose processor, a digital signal processor (“DSP”), an application specific integrated circuit (“ASIC”), a field programmable gate array (“FPGA”) or other programmable logic device, discrete gate or transistor logic, discrete hardware components, or any combination thereof designed to perform the functions described herein. A general purpose processor can be a microprocessor, but in the alternative, the processor can be a controller, microcontroller, or state machine, combinations of the same, or the like. A processor can include electrical circuitry configured to process computer-executable instructions. In another embodiment, a processor includes an FPGA or other programmable devices that performs logic operations without processing computer-executable instructions. A processor can also be implemented as a combination of computing devices, for example, a combination of a DSP and a microprocessor, a plurality of microprocessors, one or more microprocessors in conjunction with a DSP core, or any other such configuration. Although described herein primarily with respect to digital technology, a processor may also include primarily analog components. For example, some, or all, of the signal processing algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, or algorithm described in connection with the embodiments disclosed herein can be embodied directly in hardware, in a software module stored in one or more memory devices and executed by one or more processors, or in a combination of the two. A software module can reside in RAM memory, flash memory, ROM memory, EPROM memory, EEPROM memory, registers, hard disk, a removable disk, a CD-ROM, or any other form of non-transitory computer-readable storage medium, media, or physical computer storage known in the art. An example storage medium can be coupled to the processor such that the processor can read information from, and write information to, the storage medium. In the alternative, the storage medium can be integral to the processor. The storage medium can be volatile or nonvolatile. The processor and the storage medium can reside in an ASIC. The ASIC can reside in a user terminal. In the alternative, the processor and the storage medium can reside as discrete components in a user terminal.

Conditional language such as, among others, “can,” “could,” “might” or “may,” unless specifically stated otherwise, are otherwise understood within the context as used in general to convey that certain embodiments include, while other embodiments do not include, certain features, elements and/or blocks. Thus, such conditional language is not generally intended to imply that features, elements and/or blocks are in any way required for one or more embodiments or that one or more embodiments necessarily include logic for deciding, with or without user input or prompting, whether these features, elements and/or blocks are included or are to be performed in any particular embodiment.

Disjunctive language such as the phrase “at least one of X, Y, or Z,” unless specifically stated otherwise, is otherwise understood with the context as used in general to present that an item, term, and so forth, may be either X, Y, or Z, or any combination thereof (for example, X, Y, and/or Z). Thus, such disjunctive language is not generally intended to, and should not, imply that certain embodiments require at least one of X, at least one of Y, or at least one of Z to each be present.

Any process descriptions, elements or blocks in the flow diagrams described herein and/or depicted in the attached figures should be understood as potentially representing modules, segments, or portions of code which include one or more executable instructions for implementing specific logical functions or elements in the process. Alternate implementations are included within the scope of the embodiments described herein in which elements or functions may be deleted, executed out of order from that shown, or discussed, including substantially concurrently or in reverse order, depending on the functionality involved as would be understood by those skilled in the art.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation A working in conjunction with a second processor configured to carry out recitations B and C.

It should be emphasized that many variations and modifications may be made to the above-described embodiments, the elements of which are to be understood as being among other acceptable examples. All such modifications and variations are intended to be included herein within the scope of this disclosure and protected by the following.

What is claimed is:

1. A computer-implemented method for identifying synthetic identity records, the computer-implemented method comprising:
receiving a plurality of records identifying a plurality of individuals, each of the plurality of records relating to an action or a property of at least one individual of the plurality of individuals;
receiving a request from a requesting entity to determine whether a target individual identified in at least a subset of the plurality of records refers to a real person as opposed to a synthetic identity, wherein a synthetic identity is an identity defined by a set of information that does not correspond in aggregate to a real person;
identifying, from among the plurality of records, one or more records relating to the target individual;
for each machine learning model of a plurality of machine learning models:
providing one or more attributes derived from the one or more records relating to the target individual as an input to the machine learning model; and
generating a score for the target individual based on application of the machine learning model to the attributes derived from the one or more records relating to the target individual, wherein the score represents at least one of a likelihood or confidence as determined by the machine learning model that the one or more attributes are associated with one or more synthetic identities;
generating a combined score for the target individual based on the generated score from each of the plurality of machine learning models; and
based at least in part on a comparison of the combined score to a threshold value, generating a notification to the requesting entity indicating that the target individual is a real person as opposed to a synthetic identity.

2. The computer-implemented method of claim 1, wherein the notification further includes information representing that an entity implementing the computer-implemented method (a) guarantees that the target individual is not a synthetic identity and (b) will reimburse at least a portion of losses incurred by the requesting entity in association with synthetic identity fraud connected to an account opened by the target individual with the requesting entity.

3. The computer-implemented method of claim 2 further comprising generating and storing an association between the request and an account appearing in credit records subsequent to receiving the request, wherein the association represents that the account is subject to guarantee and the reimbursing of the portion of losses incurred by the requesting entity.

4. The computer-implemented method of claim 3, wherein generating the association comprises:
generating a pool of candidate inquiry and tradeline pairs based in part on credit records in a credit bureau database, wherein an individual pair of a first inquiry and a first tradeline is included in the pool based at least in part on identification of (a) a consumer identifier in common between the first inquiry and the first tradeline, (b) an identifier of a first requesting entity for the first inquiry appearing in the first tradeline, and (c) an opening date for the first tradeline is after a date of the first inquiry but earlier than a predefined number of days after the date of the first inquiry;
forming a bipartite graph of the candidate inquiry and tradeline pairs in the pool; and
selecting a single candidate inquiry and tradeline pair from among the pool of candidate inquiry and tradeline pairs, wherein the single candidate inquiry and tradeline pair is identified as a maximum-weight matching in the bipartite graph.

5. The computer-implemented method of claim 1, wherein the request from the requesting entity includes a plurality of identity data fields provided to the requesting entity by an applicant purporting to be the target individual, wherein the one or more attributes are derived based at least in part on at least one of the plurality of identity data fields.

6. The computer-implemented method of claim 1, wherein the plurality of machine learning models include at least one supervised machine learning model and at least one unsupervised machine learning model, wherein the at least one unsupervised machine learning model provides input to the at least one supervised machine learning model.

7. The computer-implemented method of claim 1, wherein one machine learning model of the plurality of machine learning models comprises a gradient boosting model with binned attributes and monotonic constraints applied on at least a subset of the one or more attributes.

8. The computer-implemented method of claim 7, wherein the one machine learning model is configured to monitor high risk identities and identities associated with one or more of (a) severe bust outs identified from credit data or (b) a large numbers of charge-offs identified from the credit data.

9. The computer-implemented method of claim 1, wherein one machine learning model of the plurality of machine learning models comprises a one-class adversarial nets (OCAN) model, wherein the computer-implemented method further comprises training the OCAN model, wherein training the OCAN model comprises:
learning real identities from training records;
training a complementary generative adversarial network (GAN) to generate complementary samples that are in a low-density area of the real identities; and
training a discriminator to distinguish between the real identities and the complementary identities.

10. The computer-implemented method of claim 1, wherein one machine learning model of the plurality of machine learning models comprises a semi-unsupervised Deep Generative Model (SU-DGM) trained to generalize synthetic identity characteristics from a population determined to be associated with at least one type of synthetic identity fraud.

11. A computer system comprising:
an electronic data store that stores a plurality of records identifying a plurality of individuals, each of the plurality of records relating to an action or a property of at least one individual of the plurality of individuals; and
at least one physical processor configured with executable instructions that cause the at least one physical processor to:
receive a request from a requesting entity to determine whether a target individual identified in at least a subset of the plurality of records refers to a real person as opposed to a synthetic identity;
identify, from among the plurality of records, one or more records relating to the target individual;
for each machine learning model of a plurality of machine learning models:
provide one or more attributes derived from the one or more records relating to the target individual as an input to the machine learning model; and
generate a score for the target individual based on application of the machine learning model to the attributes derived from the one or more records relating to the target individual, wherein the score represents at least one of a likelihood or confidence as determined by the machine learning model that the one or more attributes are associated with one or more synthetic identities;
generate a combined score for the target individual based on the generated score from each of the plurality of machine learning models; and
based at least in part on a comparison of the combined score to a threshold value, generate a notification to the requesting entity indicating that the target individual is a real person as opposed to a synthetic identity.

12. The computer system of claim 11, wherein the notification further includes information representing that an entity that operates the computer system (a) guarantees that the target individual is not a synthetic identity and (b) will reimburse at least a portion of losses incurred by the requesting entity in association with synthetic identity fraud connected to an account opened by the target individual with the requesting entity.

13. The computer system of claim 12, wherein the executable instructions further cause the at least one physical processor to generate and store an association between the request and an account appearing in credit records subsequent to receipt of the request, wherein the association represents that the account is subject to guarantee and the reimbursing of the portion of losses incurred by the requesting entity.

14. The computer system of claim 13, wherein the executable instructions causing the at least one physical processor to generate the association comprises the executable instructions causing the at least one physical processor to:
generate a pool of candidate inquiry and tradeline pairs based in part on credit records in a credit bureau database, wherein an individual pair of a first inquiry and a first tradeline is included in the pool based at least in part on identification of (a) a consumer identifier in common between the first inquiry and the first tradeline, (b) an identifier of a first requesting entity for the first inquiry appearing in the first tradeline, and (c) an opening date for the first tradeline is after a date of the first inquiry but earlier than a predefined number of days after the date of the first inquiry;
form a bipartite graph of the candidate inquiry and tradeline pairs in the pool; and
select a single candidate inquiry and tradeline pair from among the pool of candidate inquiry and tradeline pairs, wherein the single candidate inquiry and tradeline pair is identified as a maximum-weight matching in the bipartite graph.

15. The computer system of claim 11, wherein one machine learning model of the plurality of machine learning models comprises a gradient boosting model with binned attributes and monotonic constraints applied on at least a subset of the one or more attributes.

16. The computer system of claim 15, wherein the one machine learning model is configured to monitor high risk identities and identities associated with one or more of (a) severe bust outs identified from credit data or (b) a large numbers of charge-offs identified from the credit data.

17. The computer system of claim 11, wherein one machine learning model of the plurality of machine learning models comprises a one-class adversarial nets (OCAN) model, wherein the executable instructions further cause the at least one physical processor to train the OCAN model, wherein training the OCAN model comprises:
learning real identities from training records;
training a complementary generative adversarial network (GAN) to generate complementary samples that are in a low-density area of the real identities; and
training a discriminator to distinguish between the real identities and the complementary identities.

18. The computer system of claim 11, wherein each of the one or more attributes relate to one or more of: a footprint, an establishment age, one or more relationships, or one or more behaviors.

19. The computer system of claim 11, wherein one machine learning model of the plurality of machine learning models comprises a risk propagation graph score (RPGS) model trained to propagate a risk or likelihood of fraud based on closeness of identity information between records that have one or more data fields in common, wherein closeness between two records is determined based at least in part on how many data fields are in common between the two records excluding at least one data field identified as noise.

20. The computer system of claim 19, wherein the RPGS model employs a graph connecting records based at least in part on closeness between the records, wherein the RPGS model is configured to use connections in the graph to propagate an associated synthetic identity risk score associated with a seed population along connections to neighboring records and then to further neighbors of the neighboring records iteratively with attenuation.
